(19) United States
(12) Patent Application Publication
(10) Pub. No.: US 2006/0224045 A1
(43) Pub. Date: Oct. 5, 2006

(54) Integrated access device and light source for surgical procedures

(75) Inventors: Dale Edward Whipple, East Taunton, MA (US); Timothy Beardsley, Kingston, MA (US)

Correspondence Address: LAHIVE & COCKFIELD, 28 STATE STREET, BOSTON, MA, 02109, US

(73) Assignee: DePuy Spine, Inc., Raynham, MA (US)

(21) Appl. No.: 11/096,521

(22) Filed: Mar. 31, 2005

(51) Int. Cl. A61B 1/32

(52) U.S. Cl. 600/245

(57) ABSTRACT

A surgical access system for providing access to a surgical site in a patient includes an access device defining a channel and an integrated light source for illuminating the channel. A lens focuses light produced by the integrated light source along the channel. The light source may be a light emitting diode disposed in a sidewall of the access device, an electroluminescent surface forming at least a portion of the sidewall or other suitable light source capable of being integrated with the access device. An integrated power source may be integrated with the access system for independently powering the light source.

### FIELD OF THE INVENTION

The present invention relates devices used in surgery. More particularly, the present invention relates to instrumentation and a method for the providing access and illumination for surgical sites, implements and implants.

### BACKGROUND OF THE INVENTION

In minimally invasive surgical procedures, illumination of a working space may be required to facilitate use of the surgical instruments. For example, in spinal surgery, access ports, comprising generally tubular, open-ended structures, are used to provide access to a surgical site. The access ports may require illumination at the distal end thereof to facilitate the surgical procedure.

In the current state of the art, external light sources are used to provide illumination to access ports. The external light sources are separate from the access port and generate light that is conducted through a link, such as a fiber optic cable, into the access port. However, the external light sources are unwieldy, and the link used to conduct the generated light to the access port can be cumbersome and block access by a surgeon to the port. Moreover, many current light sources used for illumination, such as Xenon or Halogen light sources, require an external power supply, which often requires an expensive capital expenditure on the part of a hospital to purchase and maintain a suitable piece of capital equipment. In addition, the external light sources can be inefficient, as the light loses some power during the conduction through the link.

### SUMMARY OF THE INVENTION

The present invention provides an illuminating surgical access device including an integrated light source for producing light. The access device defines a path or port to a surgical site and the integrated light source directs the light along the path to illuminate a surgical site accessed by the access device. A lens may be provided to focus light produced by the integrated light source along the channel. The light source may be any suitable device for producing light, including, but not limited to, a light emitting diode disposed in a sidewall of the access device, an electroluminescent surface forming at least a portion of the sidewall, a liquid crystal display (LCD), a liquid crystal plastic, an incandescent light source, a light cured polymer (LCP) lighting technology or other suitable light source capable of being integrated with the access device.

According to a first aspect of the invention, an access system for providing access to a patient during surgery is provided. The access system comprises an access device comprising at least one sidewall and defining a path therethrough forming a port for accessing the patient, and a light source integrated with the sidewall for producing at least one light beam to illuminate the port of the access device.

A power source may be provided to powering the light source. The power source may be a battery, such as a thin-film battery, a power cord designed to receive power from an outlet, or other suitable means known in the art.

According to another aspect of the invention, an access system for providing access to a patient during surgery comprises a tubular body and an integrated light source. The tubular body comprises a sidewall, an open first end, and an open second end and a path extending between the open first end and the open second end for accessing the patient. The light source is integrated with the sidewall for producing light to illuminate the open second end of the tubular body. The light source may be a light emitting diode and/or an electroluminescent surface.

According to still another aspect of the invention, a method of accessing a surgical site in a patient is provided. The method comprises the steps of providing an access device comprising at least one sidewall and defining a path therethrough forming a port for accessing the patient and a light source integrated with the sidewall for producing at least one light beam to illuminate the port of the access device, powering the light source to produce light, and directing the light through the port towards the surgical site.

### BRIEF DESCRIPTION OF THE FIGURES

These and other features and advantages of the present invention will be more fully understood by reference to the following detailed description in conjunction with the attached drawings in which like reference numerals refer to like elements through the different views. The drawings illustrate principals of the invention and, although not to scale, show relative dimensions.

FIG. 1 illustrates an illuminated access system including an integrated light source according to an embodiment of the invention.

FIG. 2 illustrates the steps involved in using the illuminated access system to dilate an incision in a patient to provide access to a surgical site according to an illustrative embodiment of the invention.

FIG. 3 illustrates an example of an expandable retractor including an integrated light source for providing access to a surgical site according to another embodiment of the invention.

FIGS. 4A-4C illustrate a surgical port including an integrated light source according to another embodiment of the invention.

FIGS. 5A-5B illustrate an illuminated access system including an electroluminescent surface forming an integrated light source according to another embodiment of the invention.

FIGS. 6A-6B illustrate an illuminated access system including at least one light emitting diode forming an integrated light source according to another embodiment of the invention.

FIG. 7 illustrates an embodiment of an integrated light source for an illuminated access device according to another embodiment of the invention.

FIG. 8 illustrates an illuminated access system of another embodiment of the invention, including the light source of FIG. 7 coupled to an interior surface of an access device to integrate the light source to the system.

FIG. 9 illustrates an illuminated access system of another embodiment of the invention including the light source of FIG. 7 coupled to an exterior surface of an access device to integrate the light source to the system.

### DETAILED DESCRIPTION OF THE INVENTION

The present invention provides an improved access system for accessing a surgical site during surgery including an integrated light source. The present invention will be described below relative to certain illustrative embodiments. Those skilled in the art will appreciate that the present invention may be implemented in a number of different applications and embodiments and is not specifically limited in its application to the particular embodiments depicted herein.

The illuminated access system of the illustrative embodiment of the invention may be used in spinal surgery, for example, during a discectomy or microdiscectomy procedure to remove damaged disc material from the spine, though one skilled in the art will recognize that the invention can be used with any surgical instrument in any surgical procedure that requires illumination. Examples of surgical procedures suitable for employing the illuminated access system of the present invention include, but are not limited to, insertion of interbody fusion devices, bone anchors, fixation devices, including rods, plates and cables, artificial disks, hip stems, artifical ligaments, trochars for gastro-intestinal work, or any procedure requiring access to a patient as well as visualization. The access system may be part of any suitable implant instrument used to provide access to a particular area of a patient's body where visualization is also needed. The access system can be used to position any suitable implant, instrument and/or other device in any suitable procedure where guidance of the implant, instrument and/or device is used. Alternatively, or in addition to providing guidance, the access system may be used to dilate a surgical incision using a set of progressively larger cannulas or an expanding cannula to provide access to a surgical site.

Referring to FIG. 1, an access system of an illustrative embodiment of the invention includes an illuminating access device 10 that provides illumination to a surgical site during performance of a surgical procedure. The illustrative access device 10 is a cannula comprising a hollow tubular body 40 suitable for insertion in and/or placement adjacent to a patient's body. The illustrative access device 10 has at least one hollow channel or lumen defining a path 42 extending from an open proximal end 41 of the access device to an open distal end 43 of the access device. The path 42 may form a working channel for accessing a surgical site adjacent to or in the vicinity of the distal end 43 of the tubular body. In the illustrative embodiment, the body 40 includes open proximal end 41 that forms a proximal port 45, and the open distal end 43 forms a distal port 47 for allowing access to the surgical site. One skilled in the art will recognize that the access device 10 may have any suitable configuration and size for providing access to an area of a body. The illustrative access device may be used for retaining soft tissue away from a surgical site and/or guiding a surgical instrument, device and/or implant, though one skilled in the art will recognize that the access device may comprise any suitable device defining a path or channel requiring illumination.

As shown, the tubular body 40 of the illustrative access device 10 is formed by a cylindrical sidewall 44, though one skilled in the art will recognize that the tubular body can have any size, shape, configuration and number of sidewalls. The access device can be any suitable device defining a port for providing access to a surgical site. The access device can have any suitable cross-section and is not limited to the cylindrical cross-section shown in the illustrative embodiments. The access device can be open or closed to define an open or closed path therethrough.

An integrated light source 50 is provided to illuminate the path 42 and/or the distal port 47 at the distal end 43 of the access device 10. The integrated light source 50 produces light 51 that is directly transmitted to the path 42 and preferably the distal port 47. The light source preferably converts electrical power to light energy, though one skilled in the art will recognize that the light source may utilize any suitable means to produce light. The light source 50 is preferably integrally formed in or directly coupled to the tubular body 40. A lens 55 may be provided to focus the light produced by the light source 50 along the path 42 towards the distal port 47.

The light source may be any suitable device for producing light, including, but not limited to, incandescent light sources, an electroluminescent surface forming at least a portion of the sidewall and solid state light sources, which employ a semiconducting material to convert electricity into light, such as a light emitting diode or light emitting polymer disposed in a sidewall of the access device, a liquid crystal display (LCD), a liquid crystal plastic and a light cured polymer (LCP) lighting technology. Any other suitable light source capable of being integrated with the access device may also be used.

A power source 60, which may be integrated with or external to the tubular body 42, powers the integrated light source 50. Examples of suitable power sources include, but are not limited to a battery pack, which may comprise replaceable or rechargeable batteries inserted in or coupled to a wall of the tubular body 40, a thin-film battery, which may be integrated with a wall of the tubular body 40, a power cable connected to a power outlet, and other suitable means known in the art. Suitable battery types include, but are not limited to Alkaline, Nickel-Cadmium, Nickel-Metal Hydride, Lithium Ion cells and others known in the art. A remote battery pack may also be used in accordance with the teachings of the invention. Suitable power sources are available from Wilson Greatbatch Technologies, Inc of Clarence, N.Y.

The use of an integrated light source 50 that is directly coupled to or integrally formed in the tubular body 40 provides significant advantages over prior means of illuminating a surgical site. Through the integrated light source 50, light is produced and immediately focused, rather than traveling from a remote light source through a light transmitter, which can increase the efficiency of the light production. The integrated light source further eliminates the need for a link between a remote light source and tube, which can be cumbersome and expensive. The integrated light source thus reduces capital expenses involved in hooking up to an external light source provided by a hospital or other setting. In addition, the use of a self-contained light source allows the access system 10 to operate independently of an external power source.

The access device 10 can be spaced from or directly interface with a surgical site. The distal end 47 of the tubular body can be configured to interface with bone or another feature to facilitate positioning of the tubular body along a suitable trajectory relative to the surgical site. For example, the distal end 47 may shaped to engage a part of the surgical site, such as a vertebral structure, and can optionally include teeth or other suitable feature formed on an outer surface for engaging a part of the surgical site, such as a vertebra.

The tubular body 40 of the illustrative access device can be rigid, semi-rigid or flexible, and can have any suitable size, shape and configuration suitable for defining a working channel and/or access to a surgical site. In the illustrative embodiment, the tubular body is straight to define a straight channel therethrough, though one skilled in the art will recognize that the tubular body may define a shaped trajectory therethrough. The tubular body is not limited to a tubular structure having closed sidewalls and can be any component that defines a path, including an open channel or a solid member.

The access device can be formed of any suitable surgical material, such as, but not limited to, plastic, surgical stainless steel and other materials known in the art.

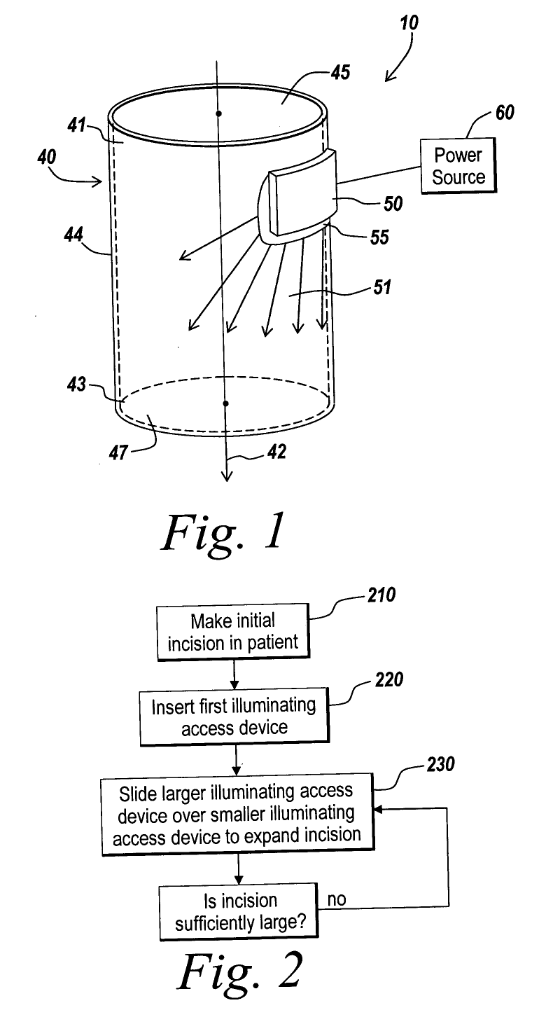

According to one embodiment of the invention, the access system comprises a plurality of illuminating access devices 10, each having a different size to allow a surgeon to expand an initial incision into the patient. FIG. 2 illustrates the steps involved in using a set of illuminating access devices to dilate an incision to provide access to a patient according to one embodiment of the invention. In step 210, an initial incision into the patient is made. In step 220, a first illuminating access device having a first, relatively narrow, diameter is inserted through the initial incision to retain the tissue surrounding the initial incision. In step 230, a larger illuminating access device is slid over the narrower illuminating access device to expand the incision. Step 230 may be repeated so that a plurality of progressively larger illuminating access devices may be sequentially slid over each other to create a working channel to the vertebra and progressively dilate the opening in the patient.

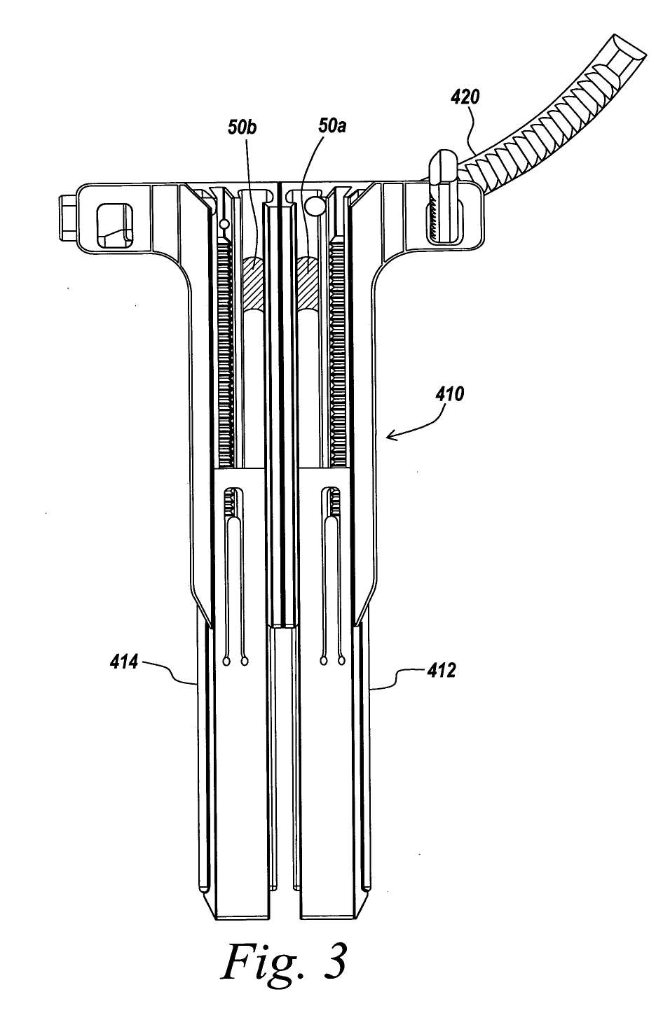

According to another embodiment of the invention, shown in FIG. 3, the access device may comprise an expandable retractor 410 having an integrated light source 50 therein. The expandable retractor has an initial tube-like configuration that splits to form blades 412, 414 that expand to push tissue apart or an organ aside to provide access to a surgical site. In the illustrative embodiment, each blade 412, 414 includes an integrated light source 50a, 50b, though one skilled in the art will recognize that the expandable retractor may have one or any suitable number of integrated light sources for illuminating a path provided by the expandable retractor 410 and/or a surgical site accessed by the expandable retractor 410. The integrated light source(s) may be provided in any suitable location within or coupled to the retractor. An actuator (not shown) selectively adjusts the position of the blades 412, 414 relative to each other during surgery to selectively expand an incision. The illustrative expandable retractor 410 may be connected to a base or other suitable system via an arm 420, which allows for positioning of the expandable retractor 410 relative to a selected surgical site.

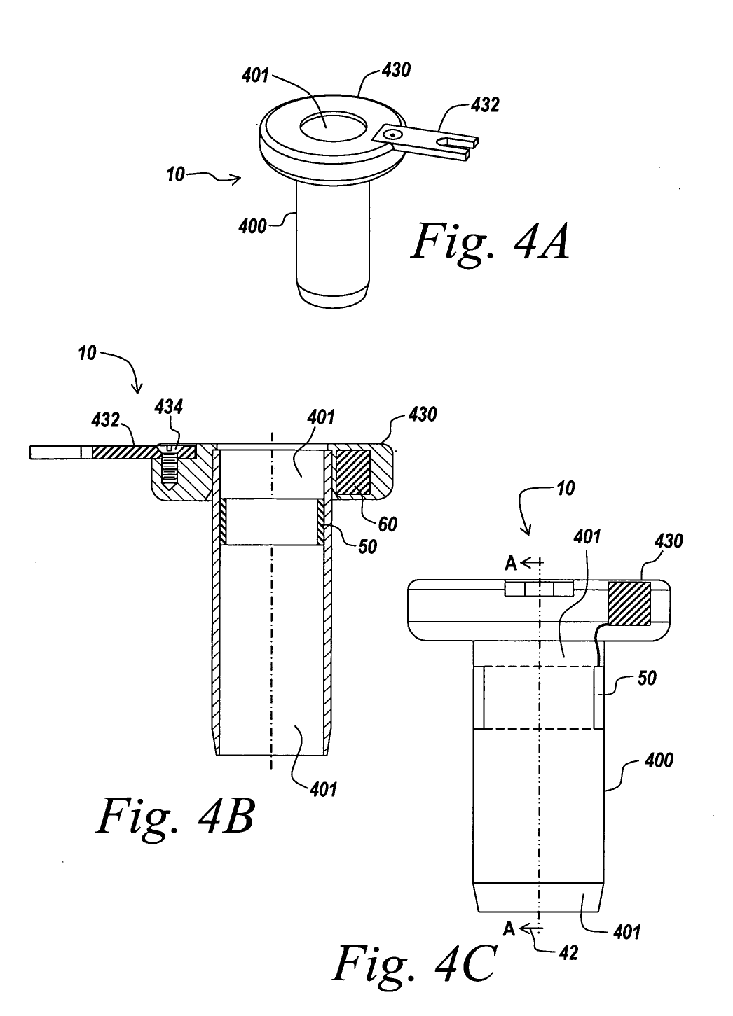

The path through the access device may also or alternatively form a working channel configured to receive and guide selected surgical instruments, such as awls, bone taps, obturators, drills, guide wires, and/or implants, such as screws, fusion devices, artificial disks and hip stems, along the longitudinal axis thereof. For example, as shown in FIGS. 4A-4B, the access system 10 may be a surgical port comprising a tubular body 400 mounted to an annular base 430. The illustrative annular base 430 includes an arm 432 for attaching the tubular body 400 to a base or other suitable system for allowing positioning of the surgical port relative to a selected surgical site. As show, a fastening device 434 is provided for coupling the base 430 to the arm 432, though one skilled in the art will recognize that any suitable means may be used for coupling the tubular body to a base or other system may be used.

The illustrative port shown in FIGS. 4A-4C may be used after serial dilation of a surgical site using a dilator. After final serial dilation is complete, a port 400 having a suitable diameter and length is inserted by sliding the port over the outer diameter of the dilator. Once the port 400 is in place, the port is secured in a selected position using the arm 432 and an attached fixation assembly, and the dilator is removed, leaving the port 400 to provide and illuminate a working channel to the surgical site.

In the embodiment shown in FIGS. 4A-4C, an integrated light source 50 is provided on the interior of the tubular body 400 for illuminating the channel 401 extending through the tubular body 400 during a subsequent surgical procedure. A power source 60 may be disposed in the base 430 to provide power to the light source 50 and ensure that the access system 10 can operate independent of external power. One skilled in the art will recognize that the integrated light source 50 and power source 60 may be provided in any suitable location and have any suitable configuration for producing light and power for producing the light, respectively.

When forming an illuminated working channel for instruments, the illuminated access device 10 preferably prevents the instruments from moving in any direction other than along a trajectory defined by the longitudinal axis 42. For guiding instruments along the working channel, the tubular body can have any suitable diameter suitable for guiding an instrument along a path defined by the tubular body. According to one embodiment, the tubular body can be configured to receive an instrument within the channel. In this embodiment, the inner diameter of the tubular body is slightly larger than the outer diameter of the instrument guided by the tubular body, so that the instrument can be inserted through the tubular body while the sidewalls of the tubular body maintain the instrument at a predetermined angle relative to the patient. Alternatively, an instrument to be guided by the tubular body is configured to slide over the tubular body, with the tubular body maintaining the orientation of the instrument as the instrument slides relative to the tubular body. In this embodiment, the tubular body can have an outer diameter that is slightly less than an inner diameter of an instrument. One skilled in the art will recognize that the tubular body can have any suitable size and configuration for guiding an instrument along a selected trajectory.

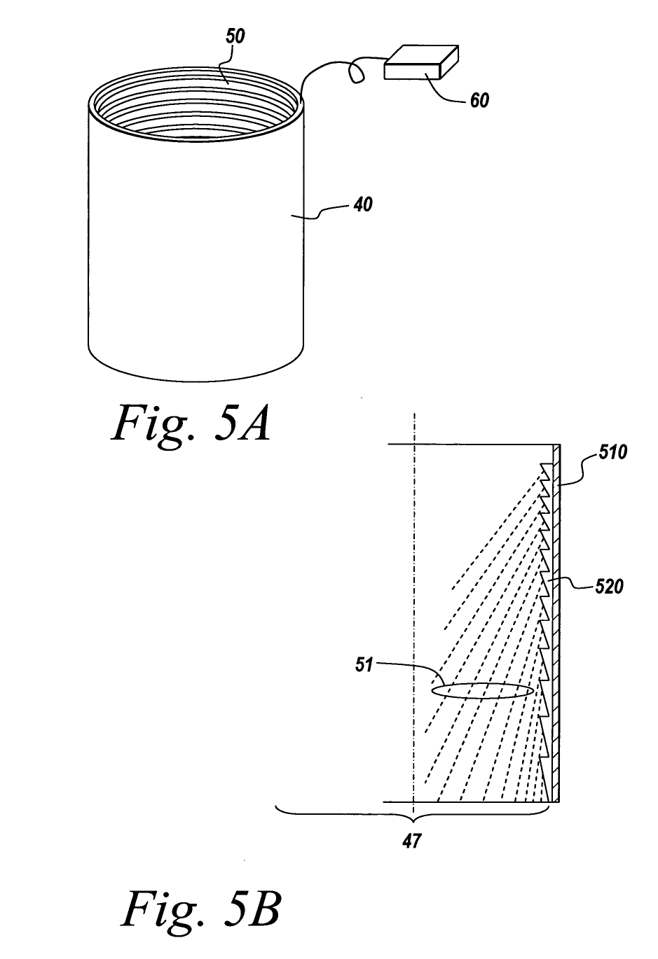

The integrated light source 50 in the surgical access system of the illustrative embodiment of the invention can comprise any suitable means for producing light that may directly coupled to or integrated with the tubular body 40 of an access device. For example, as shown in FIGS. 5A-5B, the light source 50 may comprise an electroluminescent surface 510 disposed on the inside of the tubular body to provide focused light to the interior of the tube. The electroluminescent surface 510 may by integrally formed with the sidewall defining the tubular body or layered over the sidewall on the inner surface of the tubular body. A cylindrical focusing lens 520 is provided over the electroluminescent surface 510 to focus the light 51 produced by the electroluminescent surface 510 in a selected direction. As shown, the planar illumination on inside surface of the tubular body 40 can be optically directed by the lens 520 to the distal end 43, allowing the light to be focused on a target surface, such as a surgical site, via the distal port 47.

In one embodiment, the electroluminescent surface 510 is laminated to the sidewall 44 of the tubular body, and the laminate forms the lens 520 for focusing the light produced by the electroluminescent surface 510.

The electroluminescent surface 510 may cover the entire inner surface of the tubular body 40 or a selected portion thereof. The electroluminescent surface 510 may be a unitary, continuous surface, or comprise a plurality of separate electroluminescent surfaces disposed in different locations of the access device.

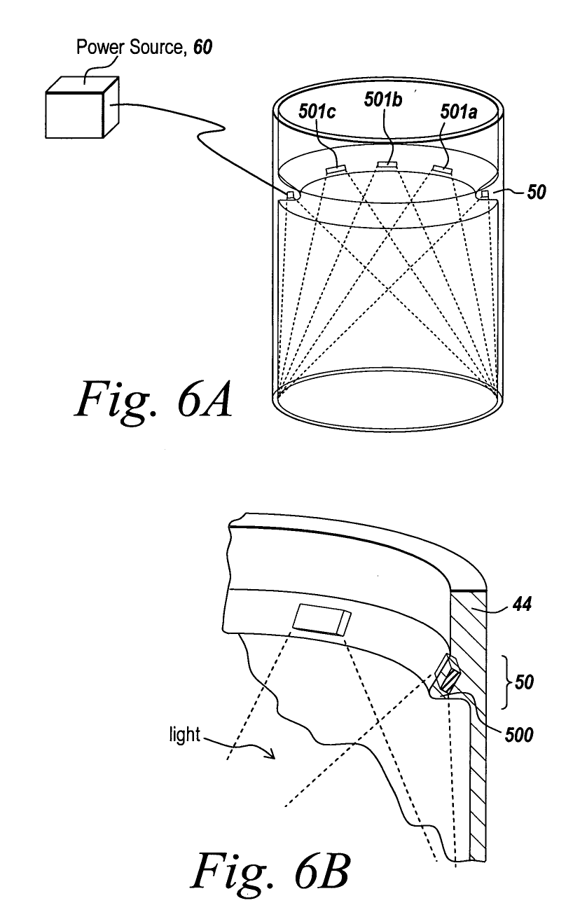

According to another embodiment of the invention, shown in FIGS. 6A-6B, the light source of an illuminated access device may comprise a solid state lighting source, illustrated as one or more light emitting diodes (LEDs), embedded in the sidewall 44 of the tubular body 40. As shown, the tubular body 40 can comprise a substantially transparent plastic tube including a shaped annular lens 500 forming a ridge on the inner surface thereof. The illustrative ridge forming the annular lens 500 is molded integrally from the sidewall 44, though one skilled in the art will recognize that the lens is not limited to the illustrative embodiment and may have any suitable configuration. The shaped annular lens 500 includes multiple LEDs 501a, 501b, 501c embedded circumferentially around the inner surface of the tube. Each LED produces light 51, which is directed by the lens 500 through the tubular body 40, towards and out the distal port 47.

While the illustrative device includes a single ring of LEDs extending around the circumference of the tubular body, one skilled in the art will recognize that the access device can include multiple rings of LEDs and lenses at different heights within the tubular body 40. In addition, the LEDs are not limited to a circumferential pattern. Rather, the light source 50 of the embodiment shown in FIGS. 6A-6B can comprise any suitable number and arrangement of LEDs integrated with the sidewall of the tubular body. Alternatively, the light source may be a light emitting polymer (LEP) or other type of solid-state light source, or an incandescent light source.

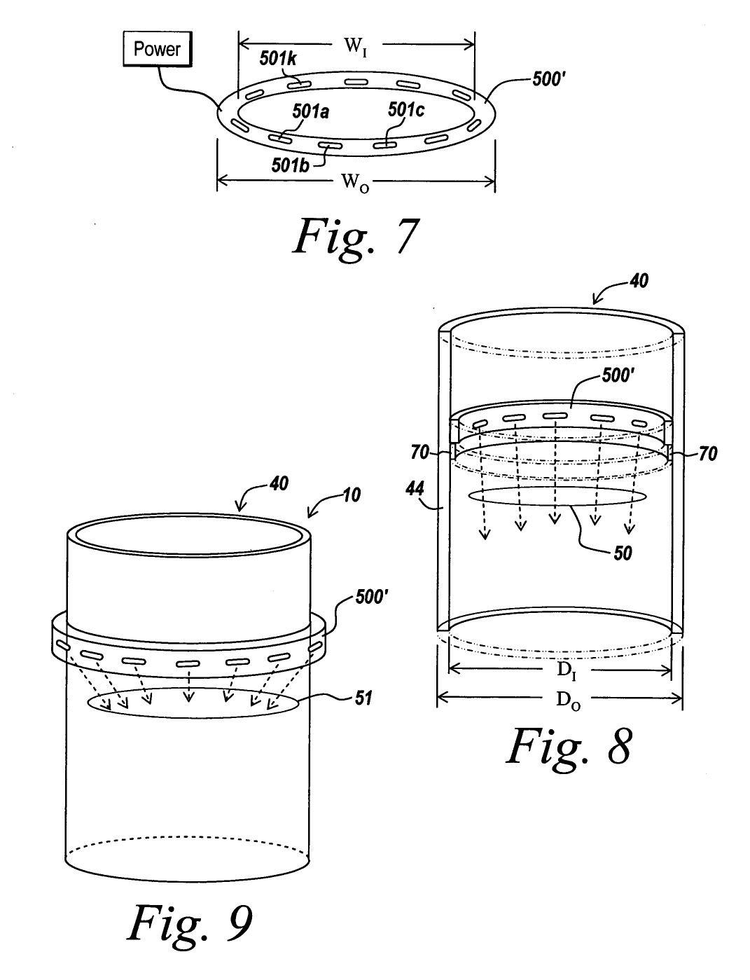

According to another embodiment of the invention, the light source 50 may comprise a molded plastic ring 500′ including embedded LEDs 501, as shown in FIG. 7. The light produced by the embedded LEDs may be focused by the plastic body of the molded plastic ring, which forms a focusing lens. Alternatively, a separate lens may be provided to focus the light produced by a LED 501.

The light ring 500′ may be configured to couple to the tubular body of the access device 10 through any suitable means. For example, the light ring 500′ be configured to couple to the inner surface of the tubular body 40, as shown in FIG. 8. In the embodiment of FIG. 8, the outer diameter Wₒ of the light ring 500′ is approximately equal to or slightly smaller than the inner diameter D_{I} of the tubular body 40. The inner surface of the tubular body may be configured to receive and retain the light ring 500′ therein. For example, in the embodiment of FIG. 8, the inner surface includes a stop 70 configured to abut the light ring 500′ and couple the light ring 500′ to the sidewall 44 to integrate the light source to the access device. Alternatively, the inner surface may have a recess configured to receive the light ring 500′, or other suitable coupling feature known in the art.

According to another embodiment, the light ring 500′ may be configured to couple to the outer surface of the tubular structure 40 to integrate the light source with the access device 10, as shown in FIG. 9. If the entire tubular body is not transparent, the light ring 500′ may align with a transparent portion of the sidewall to provide illumination to the interior of the tubular body. Alternatively, at least a portion of the light ring 500′ may align with openings in a non-transparent tubular body for conveying light from the light ring 500′ to the interior of the tubular body and/or the surgical site accessed by the associated tubular body. To couple the light ring 500′ to the tubular body 40, the inner diameter W_{I} should be slightly greater than the outer diameter D_{O} of the tubular body.

The light source may have any suitable configuration to allow the light source to directly couple to the tubular body to integrate the light source into the access device.

One skilled in the art will recognize that the access device may have any suitable size, shape and configuration for providing access to a surgical site.

The present invention has been described relative to an illustrative embodiment. Since certain changes may be made in the above constructions without departing from the scope of the invention, it is intended that all matter contained in the above description or shown in the accompanying drawings be interpreted as illustrative and not in a limiting sense. For example, one skilled in the art will recognize that the instrument of the illustrative embodiment of the invention is not limited to use with polyaxial screws and can be used with any suitable implant for any suitable orthopedic system.

It is also to be understood that the following claims are to cover all generic and specific features of the invention described herein, and all statements of the scope of the invention which, as a matter of language, might be said to fall therebetween.

1. An access system for providing access to a patient during surgery, comprising
an access device comprising at least one sidewall and defining a path therethrough forming a port for accessing the patient; and
a light source integrated with the sidewall for producing at least one light beam to illuminate the port of the access device.

2. The access system of claim 1, wherein the access device comprises a substantially tubular body.

3. The access system of claim 1, wherein the integrated light source is integrally formed with the sidewall.

4. The access system of claim 1, wherein the integrated light source is directly coupled to the sidewall.

5. The access system of claim 1, wherein the light source comprises an electroluminescent surface formed on an inner surface of the sidewall adjacent to the path.

6. The access system of claim 5, wherein the electroluminescent surface is integrally formed with the sidewall.

7. The access system of claim 5, wherein the electroluminescent surface is layered over the inner surface of the sidewall.

8. The access system of claim 1, wherein the light source comprises at least one light emitting diode.

9. The access system of claim 8, wherein the light source comprises a plurality of light emitting diodes arranged in a ring about the circumference of the path.

10. The access system of claim 9, wherein the light source comprises multiple rows of light emitting diodes formed in the sidewall.

11. The access system of claim 1, further comprising a focusing lens for focusing light produced by the light source along the path.

12. The access system of claim 1, wherein the light source comprises a plurality of light emitting diodes embedded in a molded plastic ridge on an inner surface of the sidewall.

13. The access system of claim 1, further comprising a power source connected to the light source for powering the light source.

14. The access system of claim 13, wherein the power source is integrated with the access device.

15. The access system of claim 1, wherein at least a portion of the sidewall is transparent.

16. The access system of claim 2, wherein the light source comprises a molded plastic ring including a plurality of light emitting diodes embedded therein, wherein the molded plastic ring is coupled directly to one of an inner surface and an outer surface of the sidewall to integrate the light source into the access device.

17. The access system of claim 1, wherein the light source comprises at least one of an incandescent light source, an electroluminescent surface, a light emitting diode, a liquid crystal display (LCD), a liquid crystal plastic and a light cured polymer (LCP).

18. An access system for providing access to a patient during surgery, comprising
a tubular body comprising a sidewall, an open first end, and an open second end and a path extending between the open first end and the open second end for accessing the patient; and
a light source comprising one of a light emitting diode and an electroluminescent surface integrated with the sidewall for producing light to illuminate the open second end of the tubular body.

19. The access system of claim 18, further comprising a lens disposed over the light source for focusing the light produced by the light source.

20. The access system of claim 19, wherein the lens comprises a plastic molded ridge extending from the sidewall of the tubular body, and the light source comprises a light emitting diode embedded in the molded plastic ridge.

21. A method of accessing a surgical site in a patient comprising the steps of:
providing an access device comprising at least one sidewall and defining a path therethrough forming a port for accessing the patient and a light source integrated with the sidewall for producing at least one light beam to illuminate the port of the access device;
powering the light source to produce light; and
directing the light through the port towards the surgical site.

22. The method of claim 21, further comprising the step of inserting the access device through an incision in the patient, such that the path forms a working channel to the surgical site.
